(19) United States
(12) Patent Application Publication
(10) Pub. No.: US 2010/0312308 A1
(43) Pub. Date: Dec. 9, 2010

(54) BILATERAL INPUT FOR AUDITORY PROSTHESIS

(75) Inventor: Peter A. Busby, Victoria (AU)

Correspondence Address: CONNOLLY BOVE LODGE & HUTZ LLP, 1875 EYE STREET, N.W., SUITE 1100, WASHINGTON, DC, 20006, US

(73) Assignee: COCHLEAR LIMITED, Lane Cove, NSW (AU)

(21) Appl. No.: 12/532,633

(22) PCT Filed: Mar. 25, 2008
(86) PCT No.: PCT/AU08/00415

§ 371 Date: Apr. 26, 2010

(30) Foreign Application Priority Data
Mar. 22, 2007 (AU) 2007901517

(51) Int. Cl. A61F 11/04

(52) U.S. Cl. 607/57

(57) ABSTRACT

Providing stimulation signals for an implanted auditory prosthesis including receiving first and second sound signals at first and second signal receiving devices, each of the first and second signals having a signal-to-noise ratio; determining a signal parameter related to said signal-to-noise ratio of each of the first and second signals; selecting one of the first and second signals which has the greater signal-to-noise ratio; and generating stimulation signals for the implanted auditory prosthesis based on said selected sound signal.

### CROSS-REFERENCE TO RELATED APPLICATIONS

This application is a national stage application under 35 USC §371 (c) of PCT Application No. PCT/AU2008/00415, entitled “Bilateral Input For Auditory Prostheses,” filed on Mar. 25, 2008, which claims priority from Australian Patent Application No. 2007901517, filed on Mar. 22, 2007. The entire disclosure and contents of the above applications are hereby incorporated by reference herein.

### BACKGROUND

1. Field of the Invention

The present invention relates generally to auditory prostheses, and more particularly, to audio signals for auditory prostheses.

2. Related Art

Auditory prostheses are provided to assist or replace the perception of hearing for affected individuals. Such devices include cochlear implants, middle ear implants, brain stem implants, implanted mechanical stimulators, electro-acoustic devices, and other devices which provide electrical stimulation, mechanical stimulation, or both.

In the everyday sound environment, the auditory prosthesis recipient listens to a target sound, typically speech, in the presence of background noise. In most environments, the locations of the target sound and noise sources are not the same. For example, source of speech is often in front of the auditory prosthesis recipient as the recipient is usually looking at the person talking. On the other hand, the source(s) of noise are often on the side or other locations relative to the recipient when the recipient is facing the speaker.

Background noise interferes with speech understanding, and if the level of noise approaches that of the target signal, the auditory prosthesis recipient is unable to effectively distinguish the target sound from the noise. The signal-to-noise ratio (SNR) is one measure of this influence of noise upon the target sound signal; a high SNR implies relatively low noise while a low SNR implies a relatively high noise level.

### SUMMARY

In accordance with one aspect of the invention, a method for providing stimulation signals for an implanted auditory prosthesis, the comprising: receiving first and second sound signals at first and second sound transducer, respectively, each of the first and second signals having a signal-to-noise ratio; determining a signal parameter related to said signal-to-noise ratio of each of the first and second signals; selecting one of the first and second signals which has the greater signal-to-noise ratio; and generating stimulation signals for the implanted auditory prosthesis based on said selected sound signal.

In accordance with another aspect of the invention, an auditory prosthesis is disclosed, the auditory prosthesis comprising: a first sound transducer configured to receive a first sound signal having a first signal-to-noise ratio; a second sound transducer configured to receive a second sound signal having a second signal-to-noise ratio; processing means for determining a signal parameter related to said signal-to-noise ratio of each of the first and second sound signals, selecting one of the first and second signals which has the greater signal-to-noise ratio, and for generating stimulation signals based on the selected signal.

### BRIEF DESCRIPTION OF THE DRAWINGS

Illustrative embodiments of the present invention are described herein with reference to the accompanying figures, in which:

FIG. 1 is a plan view of an auditory prosthesis recipient in an environment in which there is a target speech signal and noise;

FIG. 2 is a schematic view of an implementation of one embodiment of the system;

FIG. 3 is a block diagram of an automatic sensitivity control arrangement;

FIG. 4 is a block diagram showing one implementation of the present invention; and

FIG. 5 is a block diagram showing an alternative implementation of the present invention.

### DETAILED DESCRIPTION

Broadly, aspects of the present invention provides an arrangement in which sound signals associated with both ears are received, and the signal from the most appropriate side is used as the basis for stimulation. It will be understood that while two signals are referenced, in some implementations there may be more than two signals, for example from multiple microphones on each side.

The nature of the stimulation signals will vary with the type of device. For an implanted mechanical stimulation device, this may be raw or modified audio data. For a cochlear implant device, it may be detailed electrode and amplitude data. For an electroacoustic device, it may be both electrical and audio data, or separate signals for each type of device.

Similarly, the signal parameter may be determined either at the first and second devices, at one of the devices, or at some other part of the system such as a separate component. For the purposes of this description and claims, the signal parameter may be any measured or determined value which is useful in determining the signal-to-noise ration of the signals. For example, the signal to noise ratio itself may be used, or some other measure of signal strength relative to noise, for example a power ratio or the like, or a measure of noise.

Suitable implementations of the present invention accordingly allow the user to have the benefit of selecting for a better audio signal.

The present invention is applicable to a wide variety of partly or fully implantable auditory prostheses, particularly those which are reliant on signals provided by a microphone or other sound transducer. These prostheses include cochlear implants, middle ear implants, brain stem implants, implanted mechanical stimulators, electro-acoustic devices, or any combination of these, and other fully or partly implanted devices. The devices may have an external processor, or may be partially implanted, with only an external microphone, or even completely implanted including the microphone or other sound transducer.

For those devices having an external device, the external device may be continuously, intermittently or occasionally in communication with the implanted device. It is applicable to both unilateral and bilateral recipients.

However, aspects of the present invention will be mainly described with reference to an implementation in which a user wears two microphones and is a recipient of a cochlear implant. It will be appreciated by those skilled in the art that many other implementations and applications are possible, with suitable modifications to the implementation described.

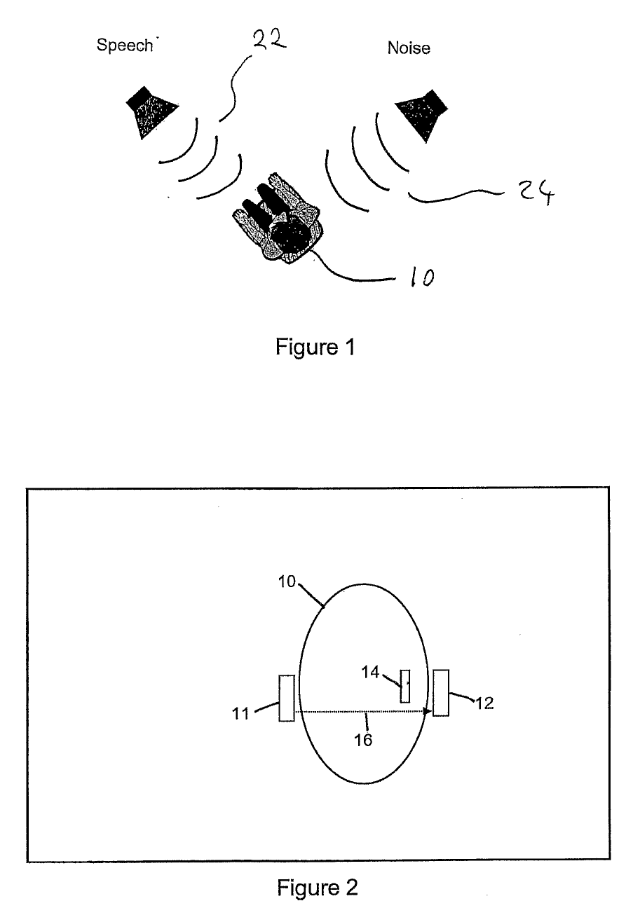

FIG. 1 shows a plan view of a typical sound environment that an auditory prosthesis recipient may encounter. The user 10 is shown seated on a chair. There is a signal that user 10 desires to hear, referred to as target signal 22. Typically, target signal 22 is a speech signal. There is also a source that generates noise signal 24.

The unilaterally implanted auditory prosthesis recipient wears the speech processor and associated microphone, which receives sound signals on the implanted ear, either the left or right. On average, the probability of noise being from the right or left side is equal in the everyday listening environment. When noise is from the same side as the microphone, the signal-to-noise ratio will be worse than if noise was from the opposite ear side. This phenomenon is known as the headshadow effect, which is essentially the attenuation of noise by the head. Bilaterally implanted users take advantage of this effect by consciously listening to the ear with the better signal-to-noise ratio, similar to a person with normal hearing. However, as described below, the present embodiment may have advantages for a bilaterally implanted recipient as well.

To take advantage of this headshadow effect, according to one implementation, the auditory prosthesis recipient wears two speech processors, with one processor situated at each ear. The two processors may be connected by cable for communication, or alternatively by radio frequency or other wireless communication method not requiring direct cable connection.

In general overview, the arrangement is shown in FIG. 2. User 10 has an implanted device 14. A processor 12 is provided on the same side as the device, which for convenience we will call the A side. However, a processor 11 is also provided on the contralateral side, which we will refer to hereafter as the B side. The two processors 11, 12 are connected by a suitable communications link 16, for example a cable. As will be described in more detail below, in this arrangement processor 12 determines whether the A or B side has experienced the best signal-to-noise ratio for the received audio signal. The output of that processor is then used as the basis for stimulation. It will be appreciated that the signal-to-noise ratio is only one particular measure which is used in the present implementation, and that the present invention can be implemented using many alternative measured or calculated parameters that are representative or indicative of the signal-to-noise ratio.

Alternatively, there may be a single speech processor and two microphones, with one microphone associated with each ear. In such a case the speech processor would process the signals from each of the microphones. It is emphasized that aspects of the present invention relates to the presence of a microphone or other transducer associated with each side of the user's head, and any suitable arrangement of processors and microphones to achieve this could be used to implement embodiments of the present invention. In particular, the microphones or other sound transducers may be external, partially or totally implanted, totally or partially in the ear canal, and associated with processors or not. In a simple implementation, the B side may be a simple microphone, connected by a cable to a speech processor on the A side.

The present invention may also be applied to a bilaterally implanted user. In this case, the selection of which signal to use can be performed by one of the speech processors, or the operations may be shared among the speech processors.

The required bandwidth and data rate for transmitting the signal depends on what data is being transmitted and the complexity of the device being used. For example, if the raw audio signal as picked up by the B side microphone is transmitted to the A side, the bandwidth will have to be large enough to cover the approximately 8 kHz of the typical cochlear implant audio frequency range at a high enough data rate . The data rate needs to be high enough to ensure that the signals from each processor are very close to being synchronized when received by the A side processor. If the delay between the signals is too large, then if the B side has the signal with the higher SNR, when the prosthesis processor on side A comes to process the transmitted signal, the speech percepts heard by the recipient will not be synchronized with the speaker's lip movements.

The signal sent from the B side may be subjected to varying levels of pre-processing. At one extreme is the transmission of raw audio data; at the other may be a fully formed set of stimulation instruction for the prosthesis. The data transmitted may be at any suitable intermediate level. In the latter case, greatly reduced bandwidth may be required.

An audio compression algorithm could be used to reduce the required bandwidth. For example, US 2006/0235490 assigned to the present applicant, the disclosure of which is hereby incorporated by reference herein, discloses a suitable coding strategy which could be applied. Other suitable commercial audio compression algorithms could also be used.

Apart from a cable arrangement, a suitable wireless arrangement could be used. As discussed above, the bandwidth required will vary with the implementation chosen. Bluetooth protocol is likely to be suitable for many applications. The person skilled in the art will be aware of the many alternatives available, and can select one with power requirements and bandwidth compatible with this use. Any suitable protocol or arrangement could be used.

It will further be appreciated that the device not specifically associated with the prosthesis may be a relatively simple device, having a microphone and a transmission arrangement to send raw audio data to the processing device, a fully functioned processor as described, or at a level of complexity and processing power in between. The processor could be separate from each of the microphone/processor devices, particularly for example in a cochlear implant which is partially or fully implanted.

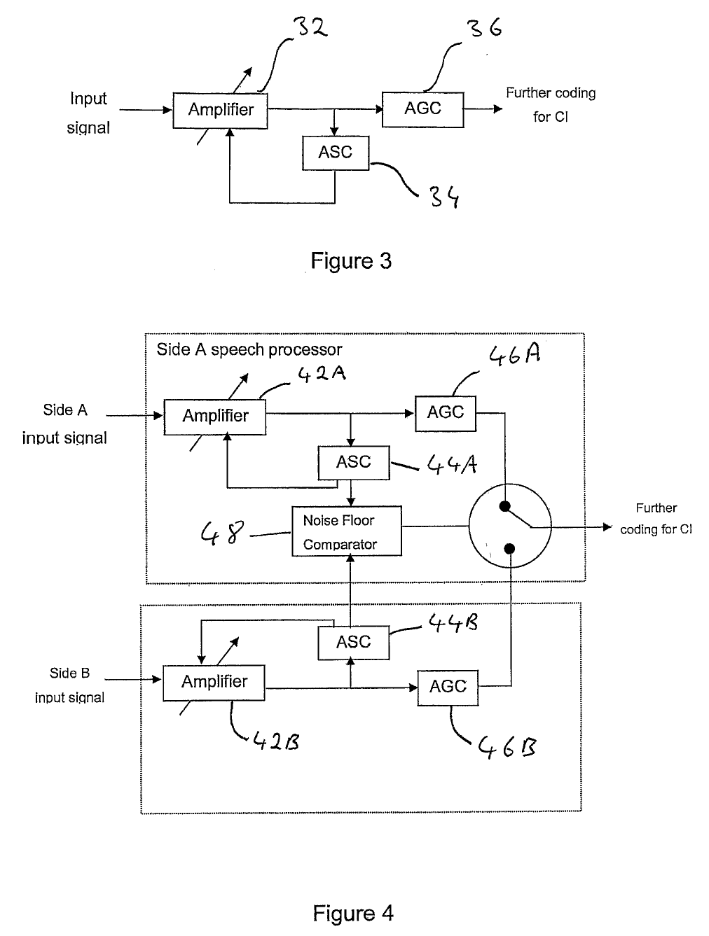

The signal-to-noise ratio at each speech processor is preferably independently measured and assessed using a suitable algorithm, for example an automatic sensitivity control (ASC) algorithm. This automatically adjusts the gain of the initial amplifier in the signal pathway, according to the level of background noise. FIG. 3 illustrates a prior art ASC arrangement, for example as described in relation to a regular, unilateral arrangement in U.S. Pat. No. 6,151,400, the disclosure of which is hereby incorporated by reference. In this arrangement, the output of the (initial) amplifier 32 is used as an input to the ASC 34. The output of the initial amplifier 32 is the input for the automatic gain control (AGC) amplifier 36. Parameters in the ASC 34 monitor the noise floor, and have pre-set breakpoint level and timing parameters. This allows the gain to be adjusted in response to the ambient noise, and hence in response to the SNR. The perceptual effect of the ASC 34 is a reduction in the loudness of background noise.

This arrangement can be applied to the A and B side signals, as shown in FIG. 4. On both the A and B side, the respective audio input signal is processed by an initial amplifier 42A, 42B, the output of which forms the input to the respective AGC 46A, 46B. Each ASC 44A, 44B receives the amplifier output, and feeds back a control signal as described. Further, according to the present implementation, a noise floor comparator 48 is provided on the A side. Each ASC 44A, 44B outputs a measure of noise floor on its respective side to the noise floor comparator 48. The characteristics of the ASCs 44A, 44B in this arrangement need to be set to be the same.

The difference between the side A and side B noise floor values is the output of the comparator 48. When the comparator 48 output value is less than or equal to the threshold value, the signal from side A is delivered to the implant. When the output value from the comparator 48 is above the threshold, the signal from side B is presented. The threshold value of the comparator 48 can be set as appropriate. The default condition is to present side A.

The noise floor comparator 48 has an adjustable time constant, typically in the order of seconds. The background noise level from each ASC 44A, 44B is preferably averaged over a time period, and this averaged value is what is used in the comparator 48. This ensures that the signal delivered to the user is not constantly changing from side to side, which could be distracting for the user.

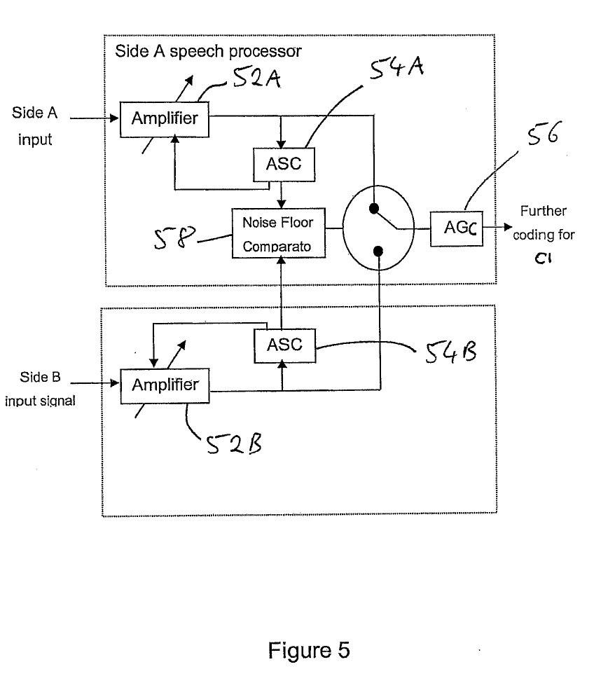

An alternative arrangement is illustrated in FIG. 5. In this arrangement, instead of each side having an AGC, the B side could be a simpler device, such as a headset microphone, including an amplifier 52B and ASC function 54B. The speech processor would perform a noise floor comparison 58, and output the selected signal to a shared AGC 56.

Any other suitable SNR assessment algorithm and control arrangement could be used, examples of which are well known to those skilled in the art. It is preferred that relatively slow time constants are used, so that that the selection program function does not switch quickly across the two processors which could be confusing for the auditory prosthesis recipient.

The A side processor according to the implementations described has the additional function of receiving from both processors the measured signal-to-noise ratio In an alternative implementation, this could be arranged to be performed in the implanted device, or elsewhere as part of the prosthesis system, although this is not presently preferred.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive. For example, whilst the present invention is described with reference to two microphones, it will be appreciated that the principal could be applied to a larger number of microphones, or to signals which are derived from microphone arrays.

1.-13. (canceled)

14. A method for providing stimulation signals for an implanted auditory prosthesis, comprising:
receiving first and second sound signals at first and second sound transducer, respectively, each of the first and second signals having a signal-to-noise ratio;
determining a signal parameter related to said signal-to-noise ratio of each of the first and second signals;
selecting one of the first and second signals which has the greater signal-to-noise ratio; and
generating stimulation signals for the implanted auditory prosthesis based on said selected sound signal.

15. An auditory prosthesis comprising:
a first sound transducer configured to receive a first sound signal having a first signal-to-noise ratio;
a second sound transducer configured to receive a second sound signal having a second signal-to-noise ratio; and
processing means for determining, for each of the first and second sound signals, a signal parameter related to the signal-to-noise ratio of the first and second sound signals, for selecting one of the first and second signals determined to have the greater signal-to-noise ratio, and for generating stimulation signals based on the selected signal.
